(19) United States
(12) Patent Application Publication
(10) Pub. No.: US 2008/0293159 A1
(43) Pub. Date: Nov. 27, 2008

(54) Validation Process

(75) Inventors: Michelle Rowney, Port Campbell (AU); Peter Hobman, Melbourne (AU); Stewart Davey, Pascoe Vale South (AU)

Correspondence Address: MYERS BIGEL SIBLEY & SAJOVEC, PO BOX 37428, RALEIGH, NC, 27627, US

(73) Assignee: Murray Goulburn Co-Operative Co. Ltd.

(21) Appl. No.: 11/782,250

(22) Filed: Jul. 24, 2007

(30) Foreign Application Priority Data
May 24, 2007 (AU) 2007902777

(51) Int. Cl. G01N 33/566

(52) U.S. Cl. 436/501

(57) ABSTRACT

The present invention provides a process for validating the efficacy of a sample having a known activity determined using a biological assay. The process includes subjecting the sample to a biological assay capable of testing for the activity. The process may optionally include an initial step of determining a biological assay for the desired activity. The present invention is particularly applicable to samples from milk.

### CROSS REFERENCE TO RELATED APPLICATION

This application claims priority to Australian Patent Application No. 2007902777, filed May 24, 2007, the disclosure of which is incorporated herein by reference in its entirety.

### FIELD OF THE INVENTION

The present invention relates to processes for the validation of the efficacy of compositions having biological activity, particularly such compositions from diary products, such as milk.

### BACKGROUND OF THE INVENTION

Milk and products generated from milk fractions are recognized to provide nutritional value. Milk is a complex mixture of molecules, including numerous polypeptides, lipids and fats, and carbohydrates. The polypeptide component, while dominated by casein, contains many other proteins having diverse functions, such as α-lactalbumin, β-lactoglobulin, immunoglobulins and caseinoglycomacropeptide.

Isolated or enriched milk components have been used as dietary supplements, often because of the ease in which the components of milk are digested. Nutraceuticals which contain milk components, particularly whey protein, have been used where the recipient desires or is in need of readily digestible protein. Applications for such nutraceuticals include body building, where it is desirable to maximize protein available for the creation of new muscle, or for administration to people who have difficulties in digesting or absorbing food normally, or those who need to gain weight.

Milk components and hydrolyzed components of milk have also been used to deliver bioactive components in edible form to subjects, such as angiotensin converting enzyme inhibiting peptides, glucagon-like peptide 1 and lactoferrin, or to reduce the risk of type 1 diabetes mellitus.

Many currently available nutraceuticals carry claims about their efficacy, which are not substantiated by clinical results. Thus, such products may not be efficacious over a placebo effect. This problem is particularly relevant for milk-derived nutraceuticals.

In Australian Patent Application No. 2006903232, the present inventors describe clinical trials, which demonstrate that a composition including a hydrolysate of whey protein is able to attenuate a reduction in muscle function, which results from muscle damage and/or is able to enhance recovery from muscle damage. This composition is a milk fraction, which was one of thousands of milk fractions tested by biological assays and shown to have a desirable biological activity. The production of this milk fraction has been scaled-up to produce the composition on a commercial scale. The scale-up process may not always produce products capable of having the desired biological activity, due to, for example, faulty enzyme batch, overheating, etc.

Accordingly, it is desirable to establish a method of validating the efficacy of a nutraceutical or composition, such as those including a milk-derived component.

### SUMMARY OF THE INVENTION

According to some aspects, the present invention provides a process for validating the efficacy of a sample having a known activity determined using a biological assay, the process includes subjecting the sample to a biological assay capable of testing for the activity.

In further aspects, the present invention provides a process for validating the efficacy of a product, the process includes: (a) determining a biological assay for a desired activity, and (b) testing a sample to validate its activity using a biological assay for the activity of (a). Steps (a) and (b) need not be carried out concurrently, or in the same site.

Additional aspects of the present invention provides a process for determining the ability of a nutraceutical composition to provide a biological activity of interest including (a) subjecting at least a portion of a component of the nutraceutical composition to a predetermined biological assay; and (b) determining the results of the biological assay, wherein a correlation between the results of the biological assay and the biological activity of interest indicates the ability of the nutraceutical composition to provide the biological activity of interest.

The present invention provides a process that is different from prior art validation processes, particularly in the pharmaceutical and food industry, where generally the composition of the sample is tested, to determine if there are contaminants. Here it is the activity of the sample that is tested thereby allowing the producers of the product to assure customers that they are purchasing a quality product.

### BRIEF DESCRIPTION OF THE DRAWINGS

In the following examples, reference will be made to the accompanying figures as follows:

FIG. 1 provides in graphical form the results of in vitro testing of test samples for their ability to influence the expression of the pro-inflammatory cytokine TNFα in a LPS stimulated macrophage cell line.

FIG. 2 provides in graphical form the results of in vitro testing of test samples for their ability to influence fibroblast cell proliferation. “FBS” represents fetal bovine serum.

### DETAILED DESCRIPTION

As used in the present specification, the singular forms “a”, “an” and “the” include plural aspects unless the context clearly dictates otherwise. Thus, for example, reference to a whey protein includes one or more whey proteins.

Also, as used herein, “and/or” refers to and encompasses any and all possible combinations of one or more of the associated listed items. Furthermore, the term “about,” as used herein when referring to a measurable value such as an amount of a compound, dose, time, temperature, and the like, is meant to encompass variations of 20%, 10%, 5%, 1%, 0.5%, or even 0.1% of the specified amount. Unless otherwise defined, all terms, including technical and scientific terms used in the description, have the same meaning as commonly understood by one of ordinary skill in the art to which this invention belongs.

All publications, patent applications, patents and other references cited herein are incorporated by reference in their entireties for the teachings relevant to the sentence and/or paragraph in which the reference is presented.

The present inventors have examined the biological activities provided by various orally administered milk fractions as set forth herein. In particular, it has been discovered that Whey Protein Isolate (WPI), a milk fraction that contains whey proteins and that may be further obtained from a variety of sources, including cheese whey and acid/casein whey, possesses an anti-inflammatory activity as identified by an in vitro assay of TNFα expression inhibition. It has further been discovered that the hydrolysis of WPI unexpectedly results in an increase in this anti-inflammatory activity. Hydrolysis of whey protein also enhanced the ability of whey protein to stimulate the growth of fibroblasts in vitro.

The present inventors have further identified that the administration of the enzyme hydrolysate of WPI is able to attenuate symptoms of muscle damage and promote the recovery of muscle function in subjects after muscle-damaging exercise. This activity is greater than the activity possessed by the corresponding non-hydrolyzed form of WPI. The hydrolysate of WPI was also able to enhance the muscle force generating ability in subjects subjected to muscle-damaging exercise.

According to particular embodiments of the present invention, a biological assay used to determine activity is used to validate efficacy. “Validating” or “validation” as used herein refers to the confirmation of a property possessed or proposed to be possessed by a sample.

“Efficacy” as used herein refers to the ability of the sample to provide the desired amount of a desired effect.

A “biological assay” includes any cellular assay as described herein and as understood those by those skilled in the art to provide assessment of a selected parameter(s).

The term “biological activity” as used herein refers to any activity of the sample on cells or tissues and includes, but is not limited to, physiological, chemical and mechanical activity as such relates to biological processes.

The term “sample” as used herein refers to a portion of a diary or milk-containing product. In particular embodiments, a sample refers to a milk fraction. The sample may be taken from an end product of the production process for the product, or may be an intermediate. The sample may also be a stored product, wherein the sample is tested to see if the product has maintained activity. The validation process may be used to determine the shelf life of the product, the effect of temperature and further processing steps, for example lyophilization, encapsulation, etc.

The present invention is particularly applicable to validating the efficacy of nutraceuticals, particularly those derived from milk. However, the present invention may be extrapolated to any product whose activity is determined using a cellular assay and whose production is not 100% guaranteed, giving the possibility that a sample of the product may not have the desired activity. This may be particularly the case if the product is produced by an enzyme or under stringent conditions.

Milk and its fractions have been proposed to have many activities, as tested by biological assays. For example, a milk fraction was described in U.S. Patent Application Publication No. 2007110818 as having COX-2 inhibitory activity as tested by assaying for inhibition of prostaglandin E2 production from HUV-EC-C cells (a permanent endothelial cell line derived from the vein of a normal human umbilical cord; ATCC CRL1730; M. Miralpeix, M. Camacho et al., Brit. J. Pharmacol. 121 (1997), 171-180). Australian patent application no. 200690303232 describes milk fractions that improve muscle function or recovery as tested by assaying for TNFα inhibition or stimulation of fibroblast cell division in vitro. WO 2007/028211 describes that WGFE decreases post-exercise inflammatory responses in muscle as tested by assaying for reduced TNFα expression in RAW cells. WO 2007/028210 describes that WGFE and WPI increase muscle strength as tested by assaying for increased myoblast cell growth and fibroblast proliferation in vitro.

In particular embodiments of the present invention, the biological assays are cell-based assays. Other biological assays may include, but are not limited to, differential gene expression and biomarker analysis, motility, chemotaxis, contraction, relaxation, biosynthesis, secretion of signaling molecules, depolarization, repolarization, degranulation, adhesion, aggregation, change in metabolic rate, and immediate cellular responses and other assays known in the art.

Other biological activities for protein fractions from milk or other sources or whole protein extracts will be known to those skilled in the art, as will assays for evaluating such biological function.

In particular embodiments, the sample is a milk fraction. Throughout the specification the term “milk fraction” refers to a composition which is ultimately derived from milk and which is at least enriched in one or more constituents found in whole milk. A “milk fraction” may be one that contains non-casein proteins found in milk. Whey or “milk plasma” is a common milk fraction which remains after the process of “curdling” has removed much of the casein and milk fat component from whole milk or skim milk. “Milk fraction” includes fractions that have been enriched for specific components or combinations of components of milk. The milk fraction “whey protein isolate” (WPI), for instance, is one in which the non-casein protein components of milk have been enriched when compared with whole milk.

“Whey protein” includes one or more protein(s) which are found in whey and which, when hydrolyzed, possesses the desired biological activity described herein. Different sources of whey protein are contemplated. In particular, whey protein originating from sweet whey or from acid whey have both been demonstrated to possess similar activity. There are, for example, numerous methods for the production of whey, for instance, as by-products arising during the production of different forms of cheese or as a by-product of the casein making process, and each of these will produce whey with a somewhat different composition. The principal proteins found in whey are α-lactalbumin and β-lactoglobulin and caseinoglycomacropeptide (CGMP), the latter forming about 25% of protein present in cheese whey WPI. Milk fractions that contain one or more of these proteins are also contemplated.

The milk may be from cows, or due to the relatively similar composition of milk possessed by domestic species, it is contemplated that milk from other animals including sheep, goats, horses and buffalo will be suitable. There may also be advantages in using whey protein of human origin, for instance in the preparation of compositions for administration to newborn babies.

The methods of the present invention are particularly useful for validating the efficacy of nutraceuticals. A “nutraceutical” as defined herein represents an edible product isolated or purified from food, which is demonstrated to have a physiological benefit or to provide protection or attenuation of an acute or chronic disease or injury when orally administered. The nutraceutical may thus be presented in the form of a dietary supplement, either alone or admixed with edible foods or drinks.

The nutraceutical composition may be in any suitable form. For example, the nutritional composition may be in the form of a soluble powder, a liquid or a ready-to-drink formulation. Alternatively, the nutritional composition may be in solid form; for example, in the form of a ready-to-eat bar, breakfast cereal or cookie, as a powder, or capsules or other form. Various flavors, fibers, sweeteners, and other additives may also be present.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

The present invention will now be described with reference to the following, non limiting examples:

### EXAMPLE 1

### Preparation of Whey Protein Isolate

The aim of the WPI process is to enrich whey proteins by removing other whey components, such as water, lactose, fat, and ash (which comprises the mineral component of milk such as phosphorous, sodium, potassium, calcium, magnesium, and other metals), until the remaining material is greater than 90% whey proteins (sample basis). Processes that are commonly used commercially to produce WPI include steps of anion exchange chromatographic separation, ultrafiltration (UF) and spray drying.

The WPI production process works on the principle of anion exchange chromatography, with a large diameter resin that allows for a high flow rates with viscous materials. The starting material may be sweet whey produced by rennet coagulation or acid whey, which results from the removal of casein by adding an acid. Whey produced by other methods, such as ultracentrifugation, microfiltration or ethanol precipitation may also be suitable. Ideally, the starting material for WPI production is WPC35 (whey protein concentrate 35% w protein/w solids), which is a generic product produced by the ultrafiltration of raw whey to remove ash and lactose. This process is widely known to those in the dairy industry. A very similar product is also produced by using skim milk instead of whey.

The starting material is introduced onto anion exchange columns (GibcoCel CR201) to load the resin with predominantly negatively charged (at the pH of whey of 6.5) whey components. The columns are rinsed with water to remove unbound materials, and the bound whey components are eluted and the columns regenerated with a mixture containing 0.75 M sodium chloride and 0.75 M potassium chloride, after which the columns are rinsed to remove residual chloride.

The whey components eluted from the CSEP are desalted and concentrated by diafiltration through low temperature, low molecular weight, spiral ultrafiltration membranes. This step retains proteins, but removes salt, ash components and lactose. During this step, total solids rise from 4% to 25% and the protein concentration rises from 50% of total solids to greater than 90% of total solids.

The purpose of the dryer is to remove the majority of the remaining water in the product until a maximum of 5% water remains. The dryer does this by atomizing the WPI concentrate in a chamber filled with hot air. As the resultant WPI powder is subsequently reconstituted in water as it is prepared for the hydrolysis, it may be possible to eliminate the drying step from this process.

The inventors have examined hydrolysates of WPI produced from cheese whey and from acid whey. Both contain similar bioactivity on enzyme digestion, and so it is likely that the type of WPI will not materially alter the resultant biological activity produced by the WPI hydrolysate.

WPI from each of sweet whey and acid whey WPI were used for the manufacture of the hydrolysates tested in vitro, but only sweet whey WPI hydrolysates were tested in vivo. The hydrolysates produced from sweet whey WPI and from acid whey WPI demonstrated very little difference in the in vitro assays, and therefore it is expected that the results for sweet whey WPI hydrolysates in vivo may be extended to acid whey hydrolysates.

### EXAMPLE 2

### Hydrolysis of WPI

The protease solution used in this example is a commercial product “Neutrase”® (Novozymes).

The target pH for the hydrolysis reaction was pH 6.5. The pH change during hydrolysis was limited, so it was practical to commence hydrolysis at pH 6.6 and not readjust the pH during the reaction. The final pH was around 6.4.

Three kilograms of WPI as prepared according to Example 1 was reconstituted in 271 water to make up 301 10% (w/v) solids solution. The pH was adjusted to 6.6 with 4M NaOH prior to heating to 50° C. Once heating had commenced, the solution was stirred continuously.

4.5 g of Neutrase® 1.5MG (Novozyme) was dissolved in 45 ml water to prepare a 10% solution, which was then added to the WPI solution. The pH was continually monitored and adjusted only if pH fell below 6.4. The hydrolysis process can be monitored in real time by the decrease in pH. The actual amount of hydrolysis that has taken place can be determined afterwards by measuring the “degree of hydrolysis” using a method based on o-phthalaldehyde (for example Lee et al., 1978; supra). The actual amount of hydrolysis taking place under the conditions described above is not very high, with an observed net degree of hydrolysis (%) between 0.3 and 3.0.

After 60 min hydrolysis, the solution was adjusted to pH 4.0 with 4M HCl and the temperature maintained at 50° C. for 30 min to deactivate the Neutrase®. The resulting hydrolysate was cooled to 25° C. and the pH readjusted ≧pH 6.5, and ideally to pH 7.0. The hydrolysate was then dried, ideally by freeze-drying at 35° C.

Hydrolysates of WPI or other milk fractions, which contain whey protein are produced using other proteinase enzymes, and their activity in inhibiting the expression of TNFα in macrophages or stimulating fibroblast cell growth in the in vitro methods described herein in Example 3 were screened. These hydrolysates are also screened for their activity in attenuating symptoms of muscle damage and in enhancing muscle contractile force in subjects using the methods described below.

### EXAMPLE 3

### Modulation of the Expression of TNFα in Macrophages and Stimulation of Fibroblast Cell Division In Vitro

### TNFα Release

RAW264.7 macrophages were seeded into 96-well plates at a density of 2×10⁴ cells/well in DMEM culture medium containing 10% heat-inactivated fetal bovine serum (FBS). Cells were then maintained in culture for 48 h at 37° C. Upon reaching the optimal cell density, the serum-containing medium was removed and cells stimulated under serum-free conditions for 6 h at 37° C. with 50 ng/ml bacterial lipopolysaccharide (Escherichia coli 055:B5) in the presence of either vehicle control (0.9% saline) or test dairy sample at 2 mg/ml. All samples contained sweetener and flavoring agent; the placebo sample contained no other compounds, the WPI sample contained WPI obtained according to the method described in Example 1, the hydrolyzed WPI samples were taken from two different Neutrase® hydrolysis batches produced according to the method described in Example 2, and the casein sample contained commercially available casein (acid casein, Murray Goulburn Co-Operative).

Following the 6 h stimulation period, levels of TNFα present in the cell-conditioned medium were measured using a highly specific TNFα sandwich ELISA and data expressed as % inhibition of TNFα release (relative to cells pre-treated with vehicle alone). Cell viability was assessed by alamarBlue staining.

### Fibroblast Growth

BalbC3T3 fibroblasts were seeded into 96-well plates at a density of 0.8×10⁴ cells/well in DMEM culture medium containing 10% fetal bovine serum (FBS) and maintained in culture overnight at 37° C. Following the overnight culture, the serum-containing medium was removed and cells stimulated in serum-free DMEM medium containing either vehicle control (0.9% saline) or test sample at 2 mg/ml. All test samples contained sweetener and flavouring agent; the placebo sample contained no other compounds, the WPI sample contained WPI produced according to the method of Example 1 and the hydrolysed WPI samples were taken from two different batches of Neutrase® hydrolysis of WPI according to the method of Example 2.

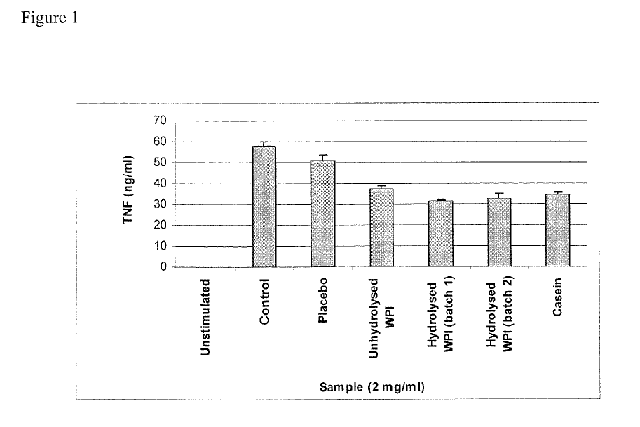

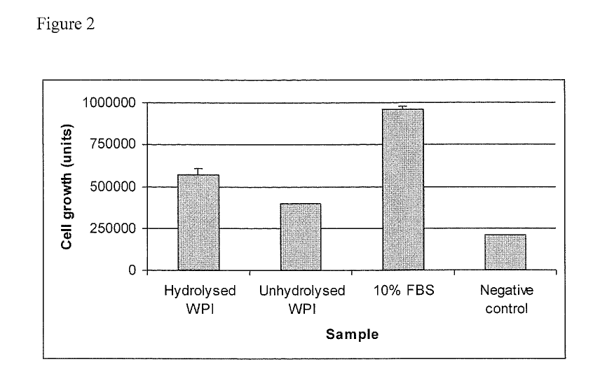

Cells were maintained in culture for an additional 48 h, after which time the culture medium was aspirated and cell growth quantitated by alamarBlue staining. Data was expressed as % growth (above cells treated with vehicle control). The results of these experiments are presented in FIGS. 1 and 2.

Of all the samples tested, the Neutrase® hydrolyzed WPI possessed the greatest activity both in inhibiting the expression of TNFα by LPS stimulated macrophages and in stimulating the growth of fibroblasts. Subsequent clinical trials showed that the Neutrase® hydrolyzed WPI was able to promote recovery from muscle damage after exercise.

### EXAMPLE 4

### Validation of Recovery Activity

Neutrase® hydrolyzed WPI was prepared as described in Examples 1 and 2 and test samples validated using the assays described in Example 3. During the optimization of process for the production of the hydrolysate, two thirds of the samples tested for activity were found not to have the desired activity and were discarded. Now that the process has been optimized, 90% of the samples tested have the required efficacy (results not shown).

1-3. (canceled)

4. The process of claim 5, wherein the portion of the component of the nutraceutical composition is milk or a fraction thereof.

5. A process for validating the efficacy of a nutraceutical composition to provide a biological activity of interest, comprising:
subjecting at least a portion of a component of the nutraceutical composition to a biological assay capable of testing for an indicator of the biological activity of interest; and
determining the results of the biological assay, wherein a correlation between the results of the biological assay and the biological activity of interest indicates the efficacy of the nutraceutical composition to provide the biological activity of interest.

6. A method of determining the ability of a product to provide a biological activity of interest, the method comprising:
subjecting a sample of the product to a biological assay capable of detecting a biological activity associated with efficacy of the product, wherein the detection of the biological activity associated with efficacy of the product indicates that the product is capable of providing a biological activity of interest compared to a product that has not been subjected to a biological assay capable of detecting said biological activity.

7. The method of claim 6, wherein the method further comprises obtaining a sample from an end product of the production process for the product or an intermediate product formed during the process of producing the product.

8. The method of claim 6, wherein the product is a nutraceutical composition.

9. The method of claim 6, wherein the sample of the product is derived from a milk product.

10. The method of claim 6, wherein the sample of the product is a milk fraction.

11. The method of claim 10, wherein the milk fraction is a whey protein isolate.

12. The method of claim 11, wherein the whey protein isolate is hydrolyzed.

13. The method of claim 6, wherein the biological activity of interest is attenuation of symptoms of muscle damage.

14. The method of claim 6, wherein the biological activity of interest is selected from the group consisting of improvement in muscle function, improvement in muscle recovery and decreased post-exercise inflammatory response.

15. The method of claim 6, wherein the biological activity of interest is enhanced muscle contractile force, increased muscle strength or cyclooxygenase-2(COX-2) inhibitory activity.

16. The method of claim 6, wherein the biological assay is an assay to determine inhibition of prostaglandin E2 production, TNFα inhibition, stimulation of fibroblast cell division, reduction of TNFα expression, increased myoblast cell growth or fibroblast proliferation.
